(19) United States
(12) Patent Application Publication
(10) Pub. No.: US 2026/0224458 A1
(43) Pub. Date: Aug. 6, 2026

(54) CLEAR GEL SUNSCREEN COMPOSITION

(71) Applicant: Kenvue Brands LLC, Summit, NJ (US)

(72) Inventors: Karlye Keller, Hoboken, NJ (US); Tatiana Luts, Bridgewater, NJ (US)

(21) Appl. No.: 19/044,999

(22) Filed: Feb. 4, 2025

(51) Int. Cl. A61K 8/35; A61K 8/04; A61K 8/25; A61K 8/37; A61K 8/40; A61K 8/891; A61K 8/92; A61Q 17/04

(52) U.S. Cl. CPC A61K 8/35; A61K 8/042; A61K 8/25; A61K 8/37; A61K 8/40; A61K 8/891; A61K 8/922; A61Q 17/04; A61K 2800/31

(57) ABSTRACT

The present invention provides a sunscreen composition comprising avobenzone; homosalate; octocrylene; octisalate; hydrogenated castor oil; silica; and dimethicone crosspolymer that is an anhydrous, clear gel having a shear viscosity of at least 20,000 cps at 25° C.

The present invention provides a sunscreen composition comprising avobenzone; homosalate; octocrylene; octisalate; hydrogenated castor oil; silica; and dimethicone crosspolymer that is an anhydrous, clear gel having a shear viscosity of at least 20,000 cps at 25° C.

### BACKGROUND OF THE INVENTION

It is well known that prolonged exposure to ultraviolet radiation, such as from the sun, can lead to the formation of light dermatoses and erythemas, and increase the risk of skin cancers, such as melanoma. Exposure to UV radiation also accelerates skin aging, such as loss of skin elasticity and wrinkling.

For these reasons, sunscreen compositions are commonly used to provide protection from the sun, and a variety of sunscreen compositions are commercially available. Sunscreen compositions typically contain a combination of organic UV absorbers such as avobenzone, homosalate, octocrylene, and octisalate, often in the form of oil-in-water emulsions to provide good aesthetic properties.

Gel sunscreen compositions are also known. They are typically single phase, anhydrous formulations that contain high levels of functionalized, specialized silicone elastomers such as Dimethicone/Bis-Isobutyl PPG-20 Crosspolymer. Unfortunately, such silicone elastomers are often single-sourced and relatively expensive. Lower cost formulations are therefore needed.

It is also challenging to formulate gel sunscreens with acceptable SPF levels that are also stable and provide desirable aesthetics, for example transparency. SPF in such products tends to be dependent on a number of factors, including choice of emollient, viscosity level, and structure.

Applicants have now discovered gel sunscreen compositions containing avobenzone, homosalate, octocrylene, and octisalate that also contain hydrogenated castor oil, silica and dimethicone crosspolymer. These compositions are anhydrous, single-phase gels that are advantageously stable, clear and have a desirable solid or semi-solid form. They also may provide in vivo SPF levels of at least about 30.

### SUMMARY OF THE INVENTION

The present invention provides a sunscreen composition comprising: (i) avobenzone; (ii) homosalate; (iii) octocrylene; (iv) octisalate; (v) at least 4.5 weight % hydrogenated castor oil based on the total weight of the composition; (vi) silica; and (vii) at least 2.1 weight % of dimethicone crosspolymer based on the total weight of the composition; wherein the composition is an anhydrous gel having a shear viscosity of at least 20,000 cps at 25° C.

The invention also provides a sunscreen composition comprising: (i) about 3 weight % of avobenzone based on the total weight of the composition; (ii) about 15 weight % of homosalate based on the total weight of the composition; (iii) about 10 weight % of octocrylene based on the total weight of the composition; (iv) about 5 weight % of octisalate based on the total weight of the composition; (v) about 5 weight % of hydrogenated castor oil based on the total weight of the composition; (vi) about 5 weight % of silica based on the total weight of the composition; and (vii) about 3.3 weight % of dimethicone crosspolymer based on the total weight of the composition; wherein the composition is an anhydrous, clear gel having a shear viscosity of about 20,000 cps at 25° C.

The invention further provides a process for preparing a sunscreen composition comprising UV absorbers, hydrogenated castor oil, silica and dimethicone crosspolymer, comprising in sequence: (i) mixing the UV absorbers and hydrogenated castor oil to form a mixture; (ii) heating the mixture to a temperature sufficient to melt the hydrogenated castor oil to form a heated mixture; (iii) decreasing the temperature of the heated mixture to a first lower temperature to form a cooled mixture; (iv) adding the silica to the cooled mixture to form a silica-containing mixture; (v) decreasing the temperature of the silica-containing mixture to a second lower temperature to form a further cooled mixture; (vi) adding the dimethicone crosspolymer to the further cooled mixture; and (vii) pouring the further cooled mixture into packaging at a temperature of at least about 35° C.

### DETAILED DESCRIPTION OF THE INVENTION

Unless defined otherwise, all technical and scientific terms used herein have the same meaning as commonly understood by one of ordinary skill in the art to which the invention belongs. All publications, patent applications, patents, and other references mentioned herein are incorporated by reference.

As used herein, “topically applying” means directly laying on or spreading on outer skin or the scalp, e.g., by use of the hands or an applicator such as a wipe, roller, or spray.

As used herein, “cosmetic” refers to a beautifying substance or preparation which preserves, restores, bestows, simulates, or enhances the appearance of bodily beauty or appears to enhance the beauty or youthfulness, specifically as it relates to the appearance of tissue or skin.

As used herein, “sunscreen composition” means a formulation that is a lotion that absorbs and/or reflects some of the sun's ultraviolet (UV) radiation and thus helps protect against negative effects of sun exposure, e.g., sunburn, premature aging, etc.

As used herein, “cosmetically effective amount” means an amount of a physiologically active compound or composition sufficient for treating one or more conditions, but low enough to avoid serious side effects. The cosmetically effective amount of the compound or composition will vary with the condition being treated, the age and physical condition of the end user, the severity of the condition being treated/prevented, the duration of the treatment, the nature of other treatments, the specific compound or product/composition employed, the cosmetically acceptable carrier utilized, and like factors.

As used herein, “cosmetically acceptable” means that the ingredients the term describes are suitable for use in contact with tissues (e.g., the skin) without undue toxicity, incompatibility, instability, irritation, allergic response, or the like.

As used herein, a “cosmetically acceptable active agent” is a compound (synthetic or natural) that has a cosmetic or therapeutic effect on the skin.

As used herein, “treatment or treating” refers to mitigating, reducing, preventing, improving, or eliminating the presence or signs of a condition or disorder.

As used herein, “substantially free of” means the ingredient referred to is not directly or intentionally added. Preferably, “substantially free of” means containing less than about 1% by weight. More preferably “substantially free of” means containing less than about 0.5% by weight. Even more preferably “substantially free of” means containing less than about 0.1% by weight. The present sunscreen composition may be completely free of an ingredient, i.e., contain none of the ingredient.

As used herein, “UV absorber” means an active ingredient whose specific function is to absorb radiation in some portion of the ultraviolet spectrum (290 nm-400 nm), such as one having an extinction coefficient of at least about 1000 mol⁻¹ cm⁻¹, for at least one wavelength within the above-defined ultraviolet spectrum. UV absorbers include for example approved sun filters and are distinguished from compounds having other functions that also happen to absorb UV radiation.

As used herein, “UV blocker” means an active ingredient whose specific function is to reflect or scatter UV radiation, for example mineral sunscreens such as titanium dioxide or zinc oxide.

As used herein, “phase-stable” means the water and oil phases of an emulsion do not appreciably separate at room temperature for at least two weeks.

Unless otherwise indicated, a percentage or concentration refers to a percentage or concentration by weight (i.e., % (W/W) based on the total weight of the composition. Unless stated otherwise, all ranges are inclusive of the endpoints, e.g., “from 4 to 9” includes the endpoints 4 and 9.

### UV Absorbers

The sunscreen composition of the invention comprises a combination of avobenzone, homosalate, octocrylene, and octisalate as UV absorbers.

Preferably, the composition is substantially free, or completely free, of oxybenzone.

The composition comprises about 2 to about 10% by weight of avobenzone based on the total weight of the composition. Preferably, the composition comprises about 3% by weight of avobenzone based on the total weight of the composition.

The composition comprises about 5 to about 15% by weight of homosalate based on the total weight of the composition. Preferably, the composition comprises about 15% by weight of homosalate based on the total weight of the composition.

The composition comprises about 5 to about 12% by weight of octocrylene based on the total weight of the composition. Preferably, the composition comprises about 10% by weight of octocrylene based on the total weight of the composition.

The composition comprises about 2 to about 10% by weight of octisalate based on the total weight of the composition. Preferably, the composition comprises about 5% by weight of octisalate based on the total weight of the composition.

In one embodiment, the composition comprises about 3% by weight avobenzone, about 15% by weight homosalate, about 10% by weight octocrylene, and about 5% by weight octisalate.

Avobenzone is commercially available, for example under the trade name Neo Heliopan 357 from Symrise.

Homosalate is commercially available, for example under the trade name Neo Heliopan HMS from Symrise.

Octocrylene is commercially available, for example under the trade name Neo Heliopan 303 from Symrise.

Octisalate is commercially available, for example under the trade name Neo Heliopan OS/BP from Symrise.

The composition need not contain additional UV absorbers or UV blockers to obtain a high SPF. Accordingly, it may be free of them. Alternatively, it may optionally contain other UV absorbers or UV blockers known in the art.

Examples of UV absorbers include, but are not limited to: methoxycinnamate derivatives such as octyl methoxycinnamate and isoamyl methoxycinnamate; camphor derivatives such as 4-methyl benzylidene camphor, camphor benzalkonium methosulfate, and terephthalylidene dicamphor sulfonic acid; other salicylate derivatives; benzoic acid derivatives such as aminobenzoic acid and octyldimethyl para-amino benzoic acid; other β,β-diphenylacrylates; dioctyl butamido triazone; octyl triazone; menthyl anthranilate; triazone derivatives such as ethylhexyl triazone (Uvinul® T150); diethylhexyl butamido triazone (UVASorb® HEB); bis-ethylhexyloxyphenol methoxyphenyl triazine (Tinosorb® S), benzoate derivatives such as diethylamino hydroxybenzoyl hexyl benzoate (Uvinul® A Plus), benzotriazole derivatives such as drometrizole trisiloxane (Mexoryl® XL), methylene bis-benzotriazolyl tetramethylbutylphenol (Tinosorb® M); tris-biphenyl triazine; (2-{4-[2-(4-Diethylamino-2-hydroxy-benzoyl)-benzoyl]-piperazine-1-carbonyl}-phenyl)-(4-diethylamino-2-hydroxy-phenyl)-methanone; phenylbenzimidazole sulfonic acid (Eusolex® 232); merocyanine derivatives; bis(butylbenzoate) diaminotriazine aminopropylsiloxane; and bis-ethylhexyloxyphenol methoxyphenyl triazine encapsulated in a polymer matrix.

UV blockers are typically inorganic metallic oxides, including titanium dioxide, zinc oxide, and certain other transition metal oxides. Such UV blockers are typically solid particles in a micronized or nanonized form having a diameter from about 0.01 micron to about 10 microns.

Similarly, the composition may optionally contain one or more polymeric organic UV absorbers. Because of their relatively high molecular weight, they are regarded as substantially non-absorbing to the skin.

As used herein, “polymeric organic UV absorber” means a polymeric compound comprising organic chromophores attached to a polymer chain. For instance, the polymer chain may be a polysiloxane having for example an average molecular weight of greater than 6000 Daltons. Examples of such polysiloxane UV absorbers include, without limitation, Parsol® SLX commercially available from DSM and polysilicone-15. These polysiloxanes absorb in the UVB (Amax=312 nm) part of the spectrum and are typically combined with UVA filters to achieve broad-spectrum protection.

### Hydrogenated Castor Oil

The composition comprises hydrogenated castor oil (Castor Wax or Hydrogenated Castor Wax). This is surprising since the composition is transparent or semi-transparent. The hydrogenated castor oil may be fully or partially hydrogenated (saturated). Preferably the hydrogenated castor oil is fully hydrogenated. Non-hydrogenated castor oil (commonly referred to as “castor oil”) is not suitable for the present invention. In addition, while variations of hydrogenated castor oil exist, such as such as “PEG-40 Hydrogenated Castor Oil” or polymers containing hydrogenated castor oil such “hydrogenated castor oil/sebacic acid copolymer,” the present invention utilizes hydrogenated castor oil rather than such variations.

The composition typically comprises about 0.5 to about 6% by weight of hydrogenated castor oil, preferably about 4 to about 5% by weight of hydrogenated castor oil, based on the total weight of the composition.

Hydrogenated castor oil is commercially available, for example, from BASF under the trade name Cutina HR Flakes.

### Dimethicone Crosspolymer

Dimethicone crosspolymer is used in the composition as a gelling agent or formula matrix.

The composition comprises at least 2.1% by weight of dimethicone crosspolymer based on the total weight of the composition. The composition may comprise about 2.7 to about 4.5% by weight, preferably about 3 to about 3.45% by weight of dimethicone crosspolymer based on the total weight of the composition. For example, the composition may comprise about 3.3% by weight of dimethicone crosspolymer based on the total weight of the composition.

Dimethicone crosspolymer is commercially available, for example, from KCC Beauty under the trade name SeraSilk EL 11 or from Dow Chemical as DOWSIL 9240. Dimethicone crosspolymer is typically provided as a blend containing 15% by weight dimethicone crosspolymer in 85% by weight dimethicone (or other vehicle).

The composition advantageously does not require the use of functionalized dimethicone crosspolymers which are expensive ingredients. Examples of these include dimethicone/bis-isobutyl PPG-20 crosspolymer, and dimethicone/PEG-10/15 crosspolymer. Accordingly, the composition may be substantially free of dimethicone/bis-isobutyl PPG-20 crosspolymer and dimethicone/PEG-10/15 crosspolymer. The composition may be completely free of dimethicone/bis-isobutyl PPG-20 crosspolymer and dimethicone/PEG-10/15 crosspolymer. Alternatively, the composition may optionally contain a dimethicone/PEG-10/15 crosspolymer, for example in an amount ranging from about 0.1 to about 3% by weight based on the total weight of the composition.

### Silica

The composition comprises silica. The composition may comprise, for example about 0.5 to about 6.5% by weight silica.

Silica is commercially available, for example, from Kobo under the trade name MSS 500/3H.

### SPF

The composition provides Sun Protection Factor (SPF) of at least about 5, preferably at least about 20, more preferably at least about 30, most preferably at least about 40. SPF may be measured in vivo or in vitro.

The SPF of the composition may be tested in vivo using the methodology set forth in the US FDA OTC monograph for sunscreens (U.S. Food and Drug Administration Over-the-Counter Monograph M020: Sunscreen Drug Products for Over-the-Counter Human Use (Posted Sep. 24, 2021); Final Administrative Order (OTC000006), effective upon enactment of the Coronavirus Aid, Relief, and Economic Security Act (CARES Act), Public Law 116-136, on Mar. 27, 2020) (“IN VIVO SPF TEST METHOD”).

The SPF of the composition may alternatively be tested using the methodology set forth in US 21 CFR § 352.76, “Determination if a product is water resistant or very water resistant” (“IN VIVO VWR SPF TEST METHOD”).

In one embodiment, the composition has an SPF as measured by the IN VIVO VWR SPF TEST METHOD of at least about 30. In another embodiment, the composition has an SPF as measured by the IN VIVO VWR SPF TEST METHOD of at least about 40.

Alternatively, the SPF of the composition may be tested using the following IN VITRO SPF TEST METHOD. The baseline transmission of a PMMA plate (substrate, available from Helioscience, Marseille, France) is measured for UV absorbance using calibrated Labsphere® UV-10005 UV transmission analyzer or a Labsphere® UV-2000S UV transmission analyzer (Labsphere, North Sutton, N.H., USA). A test sample is then applied to the PMMA plate using an application density of about 0.75 mg/cm² by rubbing into a uniform thin layer with the operator's finger. The sample is allowed to dry for 15 minutes and then measured for UV absorbance in the same way. The absorbance measures are used to calculate SPF as known in the art using the following equation:

$$SPF in vitro = \frac{{\int }_{λ = 290 n m}^{λ = 400 n m} E ( λ ) * I ( λ ) * d λ}{{\int }_{λ = 290 n m}^{λ = 400 n m} E ( λ ) * I ( λ ) * {10}^{{A}_{0} ( λ )} * d λ}$$

in which:
E(λ)=Erythema action spectrum;
I(λ)=Spectral irradiance received from the UV source;
A₀(λ)=Mean monochromatic absorbance of the test product layer before UV exposure; and
d(λ)=Wavelength step (1 nm).

### Shear Viscosity

The composition is advantageously a gel that is semi-solid or solid. For example, the composition may be in the form of a stick. The composition has a pleasing aesthetic, in that on application to the skin, it quickly liquifies.

Accordingly, it preferably has a shear viscosity of at least about 20,000 cps at 25° C. Shear viscosity is measured using a Brookfield RV, TD spindle at 4 rpm, reading at 1 minute with Helipath in the specified container, which is a 4 oz. glass jar (approx. dimensions: 4 cm diameter×10 cm height or approx. 1.5 inches×4 inches) or appropriate.

### Transparency

The composition is also transparent or semi-transparent. It is preferably invisible on application to the skin.

Transparency may be assessed using the IN VITRO WHITENESS TEST METHOD. The IN VITRO WHITENESS TEST METHOD measures the whiteness of a formula and is performed as follows. A sunscreen formulation is applied to a standardized black opacity chart with three replicates using a drawdown bar film applicator with a 6 millimeter gap. The sunscreen films are dried overnight and then the whiteness of the films are measured with a chromameter. The whitening potential of a formula is measured as the differences in the whiteness before and after the application of the sunscreen. The scale of the measurement is from 0 to 100. Formulas with a score of less than 5 are to be considered transparent or semi-transparent.

Accordingly, the sunscreen composition preferably and advantageously has a score of less than about 5 as measured by the IN VITRO WHITENESS TEST METHOD.

To compare, mineral based sunscreen lotions typically have scores of 50, and chemical based sunscreen lotions typically have scores of 12 in the IN VITRO WHITENESS TEST METHOD.

### Topical Composition

The sunscreen composition can be used by topically applying to a mammal, e.g., by the direct laying on, wiping or spreading of the composition on the skin, hair, or nails of a mammal, particularly a human.

The sunscreen composition is in the form of a solid or semi-solid that is anhydrous and single phase. The composition is stable.

The composition may be preferably prepared using stepwise mixing and cooling to provide a clear, solid or semi-solid form comprising UV absorbers, hydrogenated castor oil, silica and dimethicone crosspolymer according to the invention. The process comprises in sequence: (i) mixing the UV absorbers and hydrogenated castor oil to form a mixture; (ii) heating the mixture to a temperature sufficient to melt the hydrogenated castor oil to form a heated mixture; (iii) decreasing the temperature of the heated mixture to a first lower temperature to form a cooled mixture; (iv) adding the silica to the cooled mixture to form a silica-containing mixture; (v) decreasing the temperature of the silica-containing mixture to a second lower temperature to form a further cooled mixture; (vi) adding the dimethicone crosspolymer to the further cooled mixture; and (vii) pouring the further cooled mixture into packaging at a temperature of at least about 35° C.

Preferably, the temperature of step (ii) is about 85° C., the first lower temperature of step (iii) is about 65° C., and the second lower temperature of step (v) is about 50° C.

Preferably, emollients are added with the silica in step (iv).

A “hot pour” process is used as the final step (vii) to transfer the formulation to the desired packaging, i.e., a tube or bottle. The temperature during the step is at least about 35° C.

In a preferred embodiment, the packaging used with the sunscreen composition is airless. As used herein, “airless” means packaging that uses the force of a vacuum to push the composition out of the packaging (for example without the use of a dip tube). This prevents air exposure and oxidation, ensuring longer shelf life and maintaining the formula's integrity and stability. The airless packaging may be a bottle, jar, or tube.

Preferably, the airless packaging is a tube. More preferably, the airless packaging is an airless pump tube. An airless pump tube comprises a tube with a pump at the end of the tube, and works via a vacuum that dispenses product without letting air back in. By pressing the pump, product is expelled from the tube, which deforms (i.e., squeezes itself) and creates a vacuum, which in turn does not allow air into the tube.

Accordingly, the present invention provides a clear anhydrous gel sunscreen composition packaged in airless packaging. Preferably, the airless packaging is an airless pump tube. The sunscreen composition may comprise UV absorbers, hydrogenated castor oil, silica and dimethicone crosspolymer, and have a shear viscosity of about 20,000 cps at 25° C.

The composition may be combined with a “cosmetically acceptable topical carrier,” i.e., a carrier for topical use that capable of containing the other ingredients dispersed or dissolved therein and possessing acceptable properties rendering it safe to use topically.

The composition may optionally comprise a wide variety of additional oil-soluble materials and/or water-soluble materials conventionally used in compositions for use on skin, at their art-established levels. For example, surfactants, pearlescent or opacifying agents, thickeners, emollients, conditioners, humectants, chelating agents, exfoliants, oil absorbers, and additives that enhance the appearance, feel, or fragrance of the cleansing composition, such as colorants, fragrances, preservatives, pH adjusting agents, and the like, can be included.

In one embodiment, the composition comprises tetrasodium glutamate diacetate, a water soluble, liquid chelating agent. Surprisingly, applicants have discovered this ingredient boosts the SPF of the composition. For example, the presence of tetrasodium glutamate diacetate may increase the SPF of the composition by about 5%, preferably 15% compared with a composition containing the same ingredients without tetrasodium glutamate diacetate.

The composition may comprise, for example, about 0.5 to about 5%, preferably about 2 to about 4% by weight of tetrasodium glutamate diacetate.

Tetrasodium glutamate diacetate is commercially available, for example, from Nouryon under the trade name Dissolvine GL-47-S.

In one embodiment the composition is substantially free or, or free of, fatty alcohols. Examples of fatty alcohols include behenyl alcohol, arachidyl alcohol, stearyl alcohol and cetyl alcohol.

The sunscreen composition may also comprise film formers. Acceptable film formers include polyethylene, which is available from New Phase Technologies as Performalene® 400, a polyethylene having a molecular weight of 400. Another suitable film former is polyethylene 2000 (molecular weight of 2000), which is available from New Phase Technologies as Performalene®. Another suitable film former is synthetic wax, also available from New Phase Technologies as Performa® V-825. Other typical film-formers include VP/hexadecene copolymer, acrylates/acrylamide copolymer, acrylates copolymer, acrylates/C12-C22 alkylmethacrylate copolymer, polyethylene, waxes, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica Campestris/Aleuritis Fordi Oil copolymer, decamethyl cyclopentasiloxane (and) trimethylsiloxysilicate, Dimethicone; Acrylates/Dimethicone Copolymer; Polyester-7; Neopentyl Glycol Diheptanoate; and mixtures thereof.

The film former may for example be Polyester-7; Neopentyl Glycol Diheptanoate, for example sold from Inolex under the trade name LexFilm Sun.

The composition may comprise one or more additional oil absorbents such as aluminum starch octenylsuccinate. The composition may comprise a combination of aluminum starch octenylsuccinate or silica.

Suitable emollients for use in the composition include mineral oils, petrolatum, vegetable oils (e.g. triglycerides such as caprylic/capric triglyceride, coco-caprylate/caprate, Helianthus Annuus (Sunflower) Seed Oil), waxes and other mixtures of fatty esters, including but not limited to esters of glycerol (e.g, isopropyl palmitate, isopropyl myristate, diisopropyl adipate, dicaprylyl carbonate, c12-15 alkyl benzoate, isononyl isononanoate, neopentyl glycol diheptonoate, neopentyl glycol dibenzoate, butyloctyl salicylate, ethylhexylmethoxycrylene), hydrocarbons (e.g, isododecane, isohexadecane, squalane), ethers (e.g, PPG-15 stearyl ether, dicaprylyl ether), silicone oils such as dimethicone, and jojoba esters. In certain embodiments, mixtures of triglycerides (e.g. caprylic/capric triclycerides) and esters of glycols (e.g. isopropyl myristate) may be used.

In one embodiment, the composition comprises dimethicone. For example, the composition may comprise about 15.3 to about 25.5% by weight dimethicone based on the total weight of the composition. Dimethicone may be included in the composition as a carrier for the dimethicone crosspolymer or may be added separately to the composition.

In another embodiment, the composition comprises isododecane. For example, the composition may comprise about 0.5 to about 40% by weight isododecane based on the total weight of the composition. Isododecane is commercially available from, for example, Ineos under the trade name Permethyl 99A.

In another embodiment, the composition comprises coco-caprylate/caprate. For example, the composition may comprise about 0.5 to about 10% by weight coco-caprylate/caprate based on the total weight of the composition. Coco-caprylate/caprate is commercially available from, for example, BASF under the trade name Cetiol LC.

In a preferred embodiment, the composition contains isododecane and coco-caprylate/caprate. For example, the composition may comprise about 22 to about 35% by weight isododecane and about 3 to about 6% by weight coco-caprylate/caprateExamples of suitable solvents include propylene glycol, polyethylene glycol, polypropylene glycol, glycerol, 1,2,4-butanetriol, sorbitol esters, 1,2,6-hexanetriol, ethanol, and mixtures thereof.

In one embodiment, the sunscreen composition comprises a humectant. Examples of humectants include, without limitation to, glycerin, pentylene glycol, butylene glycol.

The composition may comprise one or more SPF boosters, for example styrene/acrylates copolymer, butyloctyl salicylate, or ethylhexyl methoxycrylene.

In one embodiment, the composition comprises ethylhexyl methoxycrylene. For example, the composition may comprise about 0.5 to about 3% by weight of ethylhexyl methoxycrylene based on the total weight of the composition. Ethylhexyl methoxycrylene is commercially available, for example, from Hallstar under the trade name SolaStay S1.

The composition may further comprise one or more other cosmetically acceptable active agents include for example anti-acne agents, shine control agents, anti-microbial agents, anti-inflammatory agents, anti-mycotic agents, anti-parasite agents, external analgesics, antioxidants, keratolytic agents, moisturizers, nutrients, vitamins, energy enhancers, anti-perspiration agents, astringents, deodorants, firming agents, anti-callous agents, and agents for skin conditioning.

The amount of other cosmetically active agents may range from about 0.001% to about 20% by weight of the composition, e.g., about 0.005% to about 10% by weight of the composition, such as about 0.01% to about 5% by weight of the composition.

The cosmetically acceptable active agent may be selected for instance from D-panthenol carotenoids, ceramides, polyunsaturated fatty acids, essential fatty acids, enzymes such as laccase, enzyme inhibitors, minerals, steroids such as hydrocortisone, 2-dimethylaminoethanol, copper salts such as copper chloride, peptides like argireline, syn-ake, acetyl dipeptide-31 amide and those containing copper, coenzyme Q10, amino acids such as proline, vitamins, lactobionic acid, acetyl-coenzyme A, niacin, riboflavin, thiamin, ribose, electron transporters such as NADH and FADH2, natural extracts such as from aloe vera, feverfew (Chrysanthemum Parthenium (Feverfew) Flower/Leaf/Steam Juice), oatmeal, dill, blackberry, princess tree, Picia anomala, and chicory, resorcinols such as 4-hexyl resorcinol, curcuminoids, sugar amines such as N-acetyl glucosamines, and derivatives and mixtures thereof.

Examples of vitamins and their derivatives include, but are not limited to, vitamin A, vitamin B's such as vitamin B3, niacinamide, vitamin B5, and vitamin B112, vitamin C, vitamin K, and different forms of vitamin E such as alpha, beta, gamma or delta tocopherols or their mixtures, and derivatives thereof.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, propolis and Zingiber Officinale (Ginger) Root Extract.

In one embodiment, the composition comprises tocopherol acetate as an antioxidant. The composition may contain, for example, about 0.01 to about 0.5% by weight tocopherol acetate. Tocopherol acetate is commercially available, for example, from BASF under the trade name DL-A-Tocopheryl Acetate.

### EXAMPLES

The following non-limiting examples further illustrate the invention.

### Example 1

A series of single-phase, oil-based, fully anhydrous sunscreen compositions were made using the ingredients shown in Table 1 (amounts in percent by weight based on the total weight of the composition) and then compared for their stability and formula structure.

The compositions were prepared by mixing and heating as follows. First, the UV filters and hydrogenated castor oil were mixed together and heated to 85° C. to melt the hydrogenated castor oil. The temperature was then decreased in a stepwise manner and the other ingredients were added as follows. The mixture was cooled to 65° C. and silica and the emollients were added. The resulting mixture was cooled to 50° C. and the dimethicone elastomer was added. Finally, the formula was transferred to a container using a “hot pour” technique at a temperature no lower than 35° C.

Stability was measured by visual inspection after 7 days at 50° C., 31 days at 40° C., 3 months at ambient room temperature (25° C.), and 3 months at 4° C. A composition was deemed “stable” if its structure was intact and free from syneresis on visual inspection.

The results are shown in Table 1. Compositions 1-5 according to the invention containing homosalate, octocrylene, octisalate, avobenzone, silica, at least 4.5 weight % of hydrogenated castor oil, and at least 2.1 weight % of dimethicone crosspolymer provided stable, gel formulations. Composition 5 provided the most desirable formula aesthetic and best overall formula stability.

TABLE 1

| | | Composition | | | | | | | | | |
|---|---|---|---|---|---|---|---|---|---|---|---|
| Ingredient | Function | 1 | 2 | 3 | 4 | 5 | 6 | 7 | 8 | 9 | 10 |
| Isododecane | Emollient | 26.9 | 22.9 | 26.4 | 34.4 | 25.9 | 31.9 | 29.9 | 34.6 | 37.9 | 33.3 |
| Homosalate | Sunscreen | 15.00 | 15.00 | 15.00 | 15.00 | 15.00 | 15.00 | 15.00 | 15.00 | 15.00 | 15.00 |
| | Active | | | | | | | | | | |
| Octocrylene | Sunscreen | 10.00 | 10.00 | 10.00 | 10.00 | 10.00 | 10.00 | 10.00 | 10.00 | 10.00 | 10.00 |
| | Active | | | | | | | | | | |
| Octisalate | Sunscreen | 5.00 | 5.00 | 5.00 | 5.00 | 5.00 | 5.00 | 5.00 | 5.00 | 5.00 | 5.00 |
| | Active | | | | | | | | | | |
| Avobenzone | Sunscreen | 3.00 | 3.00 | 3.00 | 3.00 | 3.00 | 3.00 | 3.00 | 3.00 | 3.00 | 3.00 |
| | Active | | | | | | | | | | |
| Hydrogenated | Film | 5.00 | 5.00 | 4.50 | 5.00 | 5.00 | 0.00 | 2.00 | 3.30 | 4.00 | 5.00 |
| Castor Oil | Former | | | | | | | | | | |
| Silica | Thickener | 5.00 | 5.00 | 5.00 | 5.00 | 5.00 | 5.00 | 5.00 | 5.00 | 5.00 | 6.00 |
| Tocopheryl | Antioxi- | 0.10 | 0.10 | 0.10 | 0.10 | 0.10 | 0.10 | 0.10 | 0.10 | 0.10 | 0.10 |
| Acetate | dant | | | | | | | | | | |
| Polyester-7; | Film | 3.00 | 3.00 | 3.00 | 0.00 | 3.00 | 3.00 | 3.00 | 0.00 | 0.00 | 3.00 |
| Neopentyl | Former | | | | | | | | | | |
| Glycol | | | | | | | | | | | |
| Diheptanoate | | | | | | | | | | | |
| Coco- | Emollient | 5.00 | 5.00 | 5.00 | 0.00 | 5.00 | 5.00 | 5.00 | 5.00 | 0.00 | 5.00 |
| Caprylate/ | | | | | | | | | | | |
| Caprate | | | | | | | | | | | |
| Rutin 95% | Antioxi- | 0.00 | 0.00 | 0.00 | 0.00 | 0.00 | 0.00 | 0.00 | 0.00 | 0.00 | 0.10 |
| | dant | | | | | | | | | | |
| Ethylhexyl | Skin | 0.00 | 0.00 | 1.00 | 0.00 | 1.00 | 0.00 | 0.00 | 0.00 | 0.00 | 0.00 |
| me- | Condi- | | | | | | | | | | |
| thoxycrylene | tioner | | | | | | | | | | |
| Dimethicone; | Elastomer | 0.00 | 3.00 | 0.00 | 0.00 | 0.00 | 0.00 | 0.00 | 0.00 | 0.00 | 0.00 |
| Dimethicone/ | | | | | | | | | | | |
| PEG-10/15 | | | | | | | | | | | |
| Crosspolymer | | | | | | | | | | | |
| Butylene Glycol | Humectant | 0.00 | 0.00 | 0.00 | 0.5 | 0.00 | 0.00 | 0.00 | 0.00 | 0.00 | 0.00 |
| Tertasodium | Chelating | 0.00 | 0.00 | 0.00 | 0.00 | 0.00 | 0.00 | 0.00 | 2.00 | 2.00 | 0.00 |
| Glutamate | Agent | | | | | | | | | | |
| Diacetate | | | | | | | | | | | |
| Dimethicone; | Elastomer | 22.00 (3.3 | 22.00 (3.3 | 22.00 (3.3 | 22.00 (3.3 | 22.00 (3.3 | 22.00 (3.3 | 22.00 (3.3 | 22.00 (3.3 | 18.00 (2.7 | 14.00 (2.1 |
| Dimethicone | | dimeth- | dimeth- | dimeth- | dimeth- | dimeth- | dimeth- | dimeth- | dimeth- | dimeth- | dimeth- |
| crosspolymer | | icone | icone | icone | icone | icone | icone | icone | icone | icone | icone |
| (85:15 blend) | | cross- | cross- | cross- | cross- | cross- | cross- | cross- | cross- | cross- | cross- |
| | | polymer) | polymer) | polymer) | polymer) | polymer) | polymer) | polymer) | polymer) | polymer) | polymer) |
| Formula | | Semi- | Semi- | Semi- | Semi- | Semi- | Formula | Formula | Formula | Formula | Semi- |
| Structure | | solid gel | solid gel | solid gel | solid gel | solid gel | did not | did not | did not | did not | solid gel |
| | | | | | | | create a | create a | create a | create a | |
| | | | | | | | gel | gel | gel | gel | |
| | | | | | | | structure | structure | structure | structure | |
| Stability | | Stable | Stable | Stable | Stable | Stable | Fail | Fail | Fail | Fail | Viscosity |
| | | | | | | | | | | | too low |

### Example 2

Compositions 1, 2, and 5 according to the invention were tested for SPF using the IN VIVO VWR SPF TEST METHOD. The results are shown in Table 2.

Each of the compositions had high SPF levels. Composition 5 provided the highest IN VIVO VWR SPF TEST METHOD of 40. These results show the unexpectedly high photoprotective activity of compositions according to the invention containing silica, hydrogenated castor oil, and dimethicone crosspolymer.

TABLE 2

| | Composition 1 | Composition 2 | Composition 5 |
|---|---|---|---|
| IN VIVO VWR SPF | 35 | 38 | 40 |

### Example 3

Compositions 1, 2, and 3 according to the invention were tested for transparency using the IN VITRO WHITENESS TEST METHOD. They had very low scores of 0.91, 1.29, and 3.40, respectively. These results show compositions according to the invention advantageously are transparent and non-whitening.

1. A sunscreen composition comprising:
(i) avobenzone;
(ii) homosalate;
(iii) octocrylene;
(iv) octisalate;
(v) at least 4.5 weight % hydrogenated castor oil based on the total weight of the composition;
(vi) silica; and
(vii) at least 2.1 weight % of dimethicone crosspolymer based on the total weight of the composition;
wherein the composition is an anhydrous gel having a shear viscosity of at least 20,000 cps at 25° C.

2. The sunscreen composition of claim 1 having a score of less than about 5 in the IN VITRO WHITENESS TEST METHOD.

3. The sunscreen composition of claim 1 further comprising ethylhexyl methoxycrylene.

4. The sunscreen composition of claim 1 further comprising tetrasodium glutamate diacetate.

5. A sunscreen composition comprising:
(i) about 3 weight % of avobenzone based on the total weight of the composition;
(ii) about 15 weight % of homosalate based on the total weight of the composition;
(iii) about 10 weight % of octocrylene based on the total weight of the composition;
(iv) about 5 weight % of octisalate based on the total weight of the composition;
(v) about 5 weight % of hydrogenated castor oil based on the total weight of the composition;
(vi) about 5 weight % of silica based on the total weight of the composition; and
(vii) about 3.3 weight % of dimethicone crosspolymer based on the total weight of the composition;
wherein the composition is an anhydrous, clear gel having a shear viscosity of about 20,000 cps at 25° C.

6. A process for preparing a sunscreen composition comprising UV absorbers, hydrogenated castor oil, silica and dimethicone crosspolymer, comprising in sequence:
(i) mixing the UV absorbers and hydrogenated castor oil to form a mixture;
(ii) heating the mixture to a temperature sufficient to melt the hydrogenated castor oil to form a heated mixture;
(iii) decreasing the temperature of the heated mixture to a first lower temperature to form a cooled mixture;
(iv) adding the silica to the cooled mixture to form a silica-containing mixture;
(v) decreasing the temperature of the silica-containing mixture to a second lower temperature to form a further cooled mixture;
(vi) adding the dimethicone crosspolymer to the further cooled mixture; and
(vii) pouring the further cooled mixture into packaging at a temperature of at least about 35° C.

7. The process of claim 6, wherein the temperature of step (ii) is about 85° C., the first lower temperature of step (iii) is about 65° C., and the second lower temperature of step (v) is about 50° C.

8. A clear anhydrous gel sunscreen composition packaged in airless packaging.

9. The composition of claim 8 comprising UV absorbers, hydrogenated castor oil, silica and dimethicone crosspolymer, and having a shear viscosity of about 20,000 cps at 25° C.
